(19) United States
(12) Patent Application Publication
(10) Pub. No.: US 2016/0166672 A1
(43) Pub. Date: Jun. 16, 2016

(54) Atoxic recombinant holotoxins of Clostridium difficile as immunogens

(71) Applicant: TUFTS UNIVERSITY, Boston, MA (US)

(72) Inventors: Saul Tzipori, Shrewsbury, MA (US); Xingmin Sun, Shrewsbury, MA (US)

(73) Assignee: TUFTS UNIVERSITY, Boston, MA (US)

(21) Appl. No.: 14/958,998

(22) Filed: Dec. 4, 2015

Related U.S. Application Data
(63) Continuation of application No. PCT/US2014/040993, filed on Jun. 5, 2014.
(60) Provisional application No. 61/831,543, filed on Jun. 5, 2013.

(51) Int. Cl. A61K 39/08; C12N 15/75

(52) U.S. Cl. CPC A61K 39/08; C12N 15/75; A61K 2039/53

(57) ABSTRACT

Compositions, methods and kits are provided for preparing an atoxic C. difficile protein that elicits an immune response in the subject specific for TcdA and TcdB. Atoxic Clostridium difficile toxin proteins were expressed in an endotoxin-free Bacillus system to develop a vaccine to reduce or treat incidence and severity of C. difficile infection (CDI).

### RELATED APPLICATIONS

This application claims the benefit of U.S. provisional application Ser. No. 61/831,543 filed Jun. 5, 2013 entitled, “Atoxic recombinant holotoxins of Clostridium difficile as immunogens”, inventors Saul Tzipori and Xingmin Sun which is incorporated herein in its entirety.

### GOVERNMENT SUPPORT

This invention was made with government support under grants AI088748 and DK092352 awarded by the National Institutes of Health. The government has certain rights in the invention.

### TECHNICAL FIELD

The present invention generally relates to immunogenic vaccine compositions derived from atoxic recombinant C. difficile toxin proteins and methods of making and using therefor.

### BACKGROUND

Clostridium difficile, a Gram-positive spore-forming anaerobic bacillus, is the most common cause of nosocomial antibiotic-associated diarrhea and the etiologic agent of pseudomembranous colitis (Cloud, J. et al. 2007 Curr Opin Gastroenterol 23:4-9). The disease ranges from mild diarrhea to life threatening fulminating colitis (Bartlett, J. G. 2002 N Engl J Med 346:334-339; Bordello, S. F. 1998 J Antimicrob Chemother 41 Suppl C:13-194).

C. difficile infection (CDI) is acquired by the ingestion of bacteria or bacterial spores of this strain (Dubberke, E. R. et al. 2007 Am J Infect Control 35:315-318; Roberts, K. et al. 2008 BMC Infect Dis 8:7). Spores survive contact to gastric acidity and germinate in the colon. C. difficile is the most common cause (for example about 25%) of hospital acquired and antibiotic associated diarrheas (AAD), and of almost all cases of pseudomembranous colitis (Cloud, J. et al. 2007 Curr Opin Gastroenterol 23:4-9).

Antibiotic treatment is a significant risk factor for the diseases, as are advanced age and hospitalization (Bartlett, J. G. 2006 Ann Intern Med 145:758-764). Antibiotic use permits C. difficile which is resistant to most antibiotics to proliferate and produce toxins, as post antibiotic administration the bacteria do not have to compete with the normal bacterial flora for nutrients (Owens, J. R. et al. 2008 Clinical Infectious Diseases 46:S19-S31). The toxins TcdA and TcdB are the major cause of the disease.

Interventions including administration of probiotics, toxin-absorbing polymers, and toxoid vaccines neither prevent nor treat increasing incidence and seriousness of CDI (Gerding, D. N. et al. 2008 Clin Infect Dis 46 Suppl 1:S32-42). Of further concern is the recent emergence of hyper virulent strains that are resistant to antibiotics.

The incidence of infection is increasing steadily (Archibald, L. K. et al. 2004 J Infect Dis 189:1585-1589). Several hospital outbreaks of CDI with high morbidity and mortality which occurred in the last few years in North America have been attributed to the widespread use of broad-spectrum antibiotics. The emergence of new and more virulent C. difficile strains has also contributed to the increased incidence and severity of the disease (Loo, V. G. et al. 2005 N Engl J Med 353:2442-2449; McDonald, L. C. et al. 2005 N Engl J Med 353:2433-2441). Because the surging of the incidence and severity, CDI is now considered an important emerging disease.

According to the US Agency of Healthcare Research and Quality (AHRQ), the incidence of hospital patients infected with CDI increased 200% from 2000 to 2005 and 74% increase from 1993 to 2000. Such rapid increases in incidence are attributed to usage of broad-spectrum antibiotics and/or emergence of new hyper virulent C. difficile strains. Furthermore, most cases of infection occur in patients with risk factors for antibiotic-associated colitis, and an increasing proportion of patients do not have the standard risk factors, including pregnant women, transplant patients, healthcare workers and even previously healthy people living in the community (Severe Clostridium difficile-associated disease in populations previously at low risk—four states. 2005. MMMWR 54:1201-1205).

Standard therapy includes treatment with vancomycin or metronidazole, neither of which is fully effective (Zar, F. et al. 2007 Clinical Infectious Diseases 45:302-307). An estimated 15-35% of those infected with C. difficile relapse following treatment (Barbut, F. et al. 2000 J Clin Microbiol 38:2386-2388; Tonna, I. et al. 2005 Postgrad Med J 81:367-369).

Management of CDI has been estimated to cost the US healthcare system $1.1B each year (Kuijper, E. J. et al. 2006 Clin Microbiol Infect 12 Suppl 6:2-18). The increase in rates of CDI is also associated with heightened disease severity and an increased percentage of colectomies (10.3%) and a higher mortality rate (approximately 25%) than in the past (Dallal, R. M. et al. 2002 Annals of surgery 235:363-372).

The clinical appearance of CDI infection is highly variable, from asymptomatic carriage, to mild self-limiting diarrhea, to the more severe life-threatening pseudomembranous colitis. The most common symptom is diarrhea. Other common clinical symptoms include abdominal pain and cramping, increased temperature and increase in white blood cells. In mild cases of CDI, oral rehydration plus withdrawal of antibiotics is often effective. For CDI cases that are more severe, standard therapy of oral administration of metronidazole or vancomycin is recommended, neither of which is fully effective (Zar, F. et al. 2007 Clinical Infectious Diseases 45:302-307). This treatment is also associated with a relapse rate as high as 55% (Barbut, F. et al. 2000 J Clin Microbiol 38:2386-2388; Walters, B. A. et al. 1983 Gut 24:206-212). Unfortunately, the primary treatment option for recurrent CDI remains metronidazole or vancomycin. Experimental treatments currently in clinical development include toxin-absorbing polymer, some antibiotics, and monoclonal antibodies (Anton, P. M. et al. 2004 Antimicrob Agents Chemother 48:3975-3979; Hinkson, P. L. et al. 2008 Antimicrob Agents Chemother 52:2190-2195; McVay, C. S. et al. 2000 Antimicrob Agents Chemother 44:2254-2258).

There is a need for vaccines that are easily produced and that target TcdA and TcdB to elicit strong systemic and mucosal immunity to prevent CDI, and for methods that reduce severity, eliminate ongoing chronic disease and possibly prevent relapses.

### SUMMARY

An aspect of the invention provides a composition for eliciting an immune response specific for a Clostridium difficile toxin, the composition containing an atoxic protein or a source of expression of the protein, such that the protein includes a glucosyltransferase domain (GT), a cysteine proteinase domain (CPD), a receptor binding domain (RBD), and a first amino acid sequence of the RBD derived from a TcdA protein and a second amino acid sequence of the GT and CPD from a TcdB protein, and a mutation, such that the protein is atoxic. In various embodiments, the protein is a chimeric protein.

In various embodiments of the composition, the mutation in the atoxic recombinant protein is selected from the group of the TcdA protein and TcdB protein, and the atoxic protein retains native protein conformation.

The atoxic recombinant protein in various embodiments of the composition includes the mutation in at least one C. difficile protein selected from the group of a TcdA protein and a TcdB protein. For example, the protein retains native protein conformation. In various embodiments, the toxicity of the protein is reduced at least: about 10-fold to about 1,000-fold, or about 1,000-fold to about 10,000-fold, or about 10,000-fold to about 10 million-fold compared to toxicity of wild-type Clostridium toxin. For example the toxin includes the TcdA or the TcdB.

In various embodiments of the composition, the mutation is located in the GT domain of the TcdB protein. For example, the mutation is a point mutation such as replacing a tryptophan with an alanine and replacing an aspartic acid with an asparagine. In various embodiments, the point mutations include W102A and D287N in TcdB.

In various embodiments, the atoxic recombinant protein further includes a protease cleavage site or a purification tag.

The source of the protein in various embodiments of the composition includes expressing the protein in at least one of: a Gram-positive bacterial cell; a yeast cell; a bird cell; and a mammalian cell. For example, the Gram positive bacterial cell includes a Bacillus, e.g., a B. megaterium.

In various embodiments, the composition and/or the protein is an effective dose. The composition in various embodiments further includes at least one of an adjuvant and a pharmaceutically acceptable carrier.

In various embodiments of the composition, the source of the protein includes a vector carrying a nucleotide sequence encoding the protein. For example, the vector includes a viral vector or a plasmid. In related embodiments of the composition, the viral vector is derived from a genetically engineered genome of at least one virus selected from the group consisting of adenovirus, adeno-associated virus, a herpesvirus, and a lentivirus.

The mutation in various embodiments of the composition includes at least one selected from the group consisting of: a substitution, a deletion, and an addition.

An aspect of the invention provides a method of eliciting an immune response specific for a Clostridium difficile toxin in a subject, the method including: contacting the subject with a composition containing an atoxic protein or a source of expression of the protein, such that the protein contains a glucosyltransferase domain (GT), a cysteine proteinase domain (CPD), a receptor binding domain (RBD), and a first amino acid sequence of the RBD derived from a TcdA protein and a second amino acid sequence derived of the GT and CPD from a TcdB protein, and a mutation, such that the protein is atoxic and elicits the immune response specific for the Clostridium difficile toxin in the subject. For example, the toxin includes the TcdA or the TcdB.

The method in various embodiments further includes prior to contacting, engineering a vector carrying the nucleotide sequence encoding the protein. In a related embodiment, engineering further involves expressing the protein in a cell.

In various embodiments of the method, engineering comprises obtaining the mutation in at least one of: a TcdA nucleic acid sequence encoding the TcdA amino acid sequence, and a TcdB nucleic acid sequence encoding the TcdB amino acid sequence. For example, the mutation includes at least one selected from the group consisting of: a substitution, a deletion, and an addition. For mutation is present in the nucleic acid sequence or nucleotide sequence of the gene encoding protein. Alternatively, the mutation is present in the amino acid sequence of the protein. For example, the substitution includes replacing an amino acid residue having a similar side chain. Alternatively, the substitution includes replacing an amino acid residue having a different side chain. Families of amino acid residues having similar side chains have been defined in the art. These families include for example amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine).

The source of expression of protein in various embodiments of the method is at least one selected from the group consisting of: a nucleic acid vector with a gene encoding the protein; a viral vector with a gene encoding the protein; and a cell that expresses the protein. For example, the cell is a bacterial cell, e.g., a Bacillus.

In various embodiments of the method, contacting the subject further includes administering the protein by a route selected from at least one of the group consisting of intravenous, intramuscular, intraperitoneal, intradermal, mucosal, subcutaneous, sublingual, intranasal and oral.

The method in various embodiments further includes after contacting the subject, analyzing an antibody titer in serum of the subject, and observing an increase in the titer of the antibody that specifically binds a Clostridium antigen compared to prior to control serum obtained prior to contacting, or compared to that in a control not so contacted, such that the immune response is elicited. For example, the increase in titer is at least: about one-fold to about two-fold, about two-fold to above four-fold, about four-fold to about eight-fold, about eight-fold to about ten-fold, about ten-fold to about 15-fold, about 15-fold to about 20-fold, about 20-fold to about 30-fold, about 30-fold to about 40-fold. In various embodiments, contacting effectively protected the subject from a ten-fold, a hundred-fold or a thousand-fold a lethal dose of a single disease agent such as TcdA and TcdB. In various embodiments the subject is an adult human or child.

An aspect of the invention provides a method of producing a recombinant atoxic Clostridium difficile toxin protein, the method including: constructing a vector carrying a nucleotide sequence encoding the protein, such that the protein includes a glucosyltransferase domain (GT), a cysteine proteinase domain (CPD), a receptor binding domain (RBI)), a first amino acid sequence of the RBD from a TcdA protein and a second amino acid sequence of the GT and CPD from a TcdB protein, and a mutation, such that the protein is atoxic; contacting a cell with the vector under conditions suitable to transformation or transduction of the cell; and, selecting a transformant carrying the selectable marker and expressing the recombinant atoxic Clostridium toxin protein.

In various embodiments, the cell is a protoplast selected from the group of: B. megaterium, B. subtilis, B. thuringiensis, B. cereus, and B. licheniformis. In various embodiments, the cell includes a bacterial cell, a yeast cell, a bird cell, and a mammalian cell. For example, the bacterial cell includes a Gram-positive cell.

In various embodiments, constructing the vector includes combining a first nucleic acid sequence encoding the TcdA first amino acid sequence and a second nucleic acid sequence encoding the TcdB second amino acid sequence, and such that sequence is operably linked to a regulatory region including a promoter. In various embodiments of the method, the protein includes a plurality of mutations selected from at least one of: a substitution, a deletion, and an addition. For example at least one of the mutations is located in the GT domain of the TcdB protein. The mutation of the protein in various embodiments includes a deletion of a transmembrane domain (TMD). For example, the deletion includes the TMD of TcdB protein.

An aspect of the invention provides a kit for eliciting an immune response specific against Clostridium toxin, the kit comprising: a composition including an atoxic protein or a source of expression of the protein, such that the protein contains a GT, a CPD, a RBD, and a first amino acid sequence of the RBD derived from a TcdA protein and a second amino acid sequence of the GT and CPD from a TcdB protein, and a mutation, such that the protein is atoxic; and, a container.

In various embodiments of the kit, the source of the protein includes a vector carrying a nucleotide sequence. For example, the vector includes a viral vector or a plasmid.

In various embodiments of the kit, the protein is in a unit dose suitable for an adult human subject or a child.

An aspect of the invention provides a composition for eliciting an immune response specific for Clostridium difficile toxins TcdA and TcdB, the composition including a protein or a source of expression of the protein, the protein including a GT and a CPD of the TcdB, a RBD of the TcdA, and at least two point mutations in the GT, such that the protein is atoxic. In general the atoxic protein is recombinant.

In various embodiments, the atoxic protein has reduced toxicity about 10-fold to about 1,000-fold, or about 1,000-fold to about 10,000-fold, or about 10,000-fold to about 10 million-fold compared to toxicity of wild-type Clostridium toxin.

In various embodiments, the protein includes a Tcd138 protein, a derivative, or a homolog thereof. In various embodiments, the Tcd138 protein is less toxic than protein cTxAB found in Feng et al., international application number PCT/US2010/058701 filed Dec. 2, 2010. The protein in various embodiments further includes a purification tag such as a His tag.

In various embodiments, the source of the protein includes expressing the protein in a Gram-positive bacterial cell, or in another expression system including an E. coli cell, a mammalian cell, an avian cell, or an insect cell. For example, the source of the protein comprises a recombinant cell selected from the group of: a bacterial cell, a mammalian cell, an avian cell, and an insect cell.

In a related embodiment, the Gram positive bacterial cell includes a Bacillus, or other expression system including a bacterial cell.

The composition in various embodiments is in an effective dose. In related embodiments, the composition further includes at least one of an adjuvant and a pharmaceutically acceptable carrier.

In various embodiments of the composition, the source of the protein includes a vector carrying a nucleotide sequence encoding the protein. In a related embodiment, the vector includes a plasmid. In various embodiments of the composition, the vector includes a viral vector, for example the viral vector is derived from a genetically engineered genome of at least one virus selected from the group consisting of adenovirus, adeno-associated virus, a herpesvirus, and a lentivirus.

In various embodiments of the composition, at least one mutation in the protein is one selected from the group consisting of: a substitution, a deletion, and an addition

An aspect of the invention provides a method of eliciting an immune response specific for Clostridium difficile toxins TcdA and TcdB in a subject, the method including: contacting the subject with a composition comprising an atoxic protein or a source of expression of the protein, such that the protein includes a GT and a CPD of the TcdB, a RBD of the TcdA, and at least two point mutations, such that the protein is atoxic and elicits the immune response specific for the TcdA and the TcdB in the subject.

The method in various embodiments further includes prior to contacting, engineering a vector carrying the nucleotide sequence encoding the protein. In a related embodiment of the method, engineering further includes expressing the protein in an expression system. In various embodiments of the method, engineering includes obtaining at least one mutation in TcdB nucleic acid sequence encoding the TcdB amino acid sequence. In various embodiments of the method, the at least one mutation comprises at least one selected from the group consisting of: a substitution, a deletion, and an addition.

The source of expression of protein in various embodiments is at least one selected from the group consisting of a nucleic acid vector with a gene encoding the protein, a viral vector with a gene encoding the protein; and a cell that expresses the protein. For example, the cell is a mammalian cell.

In various embodiments of the method, contacting the subject further includes administering the protein by a route selected from at least one of the group consisting of: intramuscular, subcutaneous, intraperitoneal, intradermal, sublingual, intranasal, and oral. In various embodiments, administering is performed with or without adjuvant.

The method in various embodiments further including after contacting the subject, analyzing an antibody titer in serum of the subject, and observing an increase in the titer of the antibody that specifically binds a Clostridium antigen compared to prior to control serum obtained prior to contacting, or compared to that in a control not so contacted, such that the immune response is elicited.

An aspect of the invention provides a method of producing a recombinant atoxic Clostridium difficile toxin protein, the method including: constructing a vector carrying a nucleotide sequence encoding the protein, such that the protein includes a glucosyltransferase domain (GT) and a cysteine proteinase domain (CPD) of TcdB, a receptor binding domain (RBD) of TcdA, and at least two point mutations in the GT of the TcdB, wherein protein is atoxic; transforming a cell with the vector under conditions suitable to transformation or transduction of the cell; and selecting a transformant carrying the selectable marker and expressing the recombinant atoxic Clostridium toxin protein. In various embodiments of the method, the protein is chimeric.

The cell in various embodiments of the method is a protoplast from a Bacillus, for example a B. megaterium.

In various embodiment of the method, at least one mutation is located in the GT domain of the N-terminus of the TcdB.

An aspect of the invention provides a kit for eliciting an immune response specific against Clostridium toxin, the kit including: a composition including a protein or a source of expression of the protein, the protein including a glucosyltransferase domain (GT) and a cysteine proteinase domain (CPD) of the TcdB, a receptor binding domain (RBD) of the TcdA, and at least two point mutations in the GT, such that the protein is atoxic; a container.

The source of the protein in various embodiments includes a vector carrying a nucleotide sequence. For example, the vector includes a plasmid. The source of expression of protein in various embodiments of the kit is at least one selected from the group consisting of: a nucleic acid vector with a gene encoding the protein, a viral vector with a gene encoding the protein; and a cell that expresses the protein. For example, the cell is a mammalian cell.

The protein in various embodiments is in a unit dose suitable for an adult human subject or a child.

### DETAILED DESCRIPTION

The global emergence of hyper virulent drug-resistant strains and the surge in incidence of Clostridium difficile infection (CDI) represent a major public health concern (Kelly, C P et al. 2008 N Engl J Med 359: 1932; Rupnik, M. H. et al. 2009 Nat Rev Microbiol 7: 526). C. difficile secretes two homologous glucosylating exotoxins TcdA and TcdB that are both pathogenic (Lyras D et al. 2009, Nature 458: 1176; Kuehne, S A et al. 2010 Nature), thus requiring neutralization to prevent disease occurrence.

Examples herein describe vaccines including a parenteral vaccine that induces potent neutralizing antibodies that are specific for both toxins and provide full protection against primary and recurrent CDI in mice. Using a non-pathogenic Bacillus megaterium expression system (Vary P S et al. 2007 Applied microbiology and biotechnology 76: 957; Yang, G et al. 2008 BMC Microbiol 8: 192), glucosyltranferase (GT)-deficient holotoxins were generated and absence of toxicity was demonstrated. The native form of atoxic holotoxin induced significantly more potent anti-toxin neutralizing antibodies than the corresponding toxoid. There was little cross-immunogenicity between TcdA and TcdB. To induce antibodies against both toxins, a clostridial toxin-like chimeric protein in various embodiments was designed by replacing the receptor binding domain of TcdB with that of TcdA and the GT-deficient form was generated and designated cTxAB. Parenteral immunization with this single antigen cTxAB was observed in Examples herein to induce rapid and potent neutralizing antibodies specific for both TcdA and TcdB, conferring complete protection against CDI of both a laboratory and a hyper virulent strain. A murine CDI relapse model was established that showed that this vaccine conferred rapid protection both for primary and recurrent C. difficile infection, thus providing a suitable potential prophylactic vaccine for individuals at high risk of developing CDI.

Clostridium difficile TcdA and TcdB are glucosyltransferases (GT) having an ability to modify host Rho family proteins that cause the primary virulent factor. Serum antibodies specific for the two toxins are associated with protection in patients (Kyne, L et al. 2001 Lancet 357: 189; B. A. Leav, B A et al. 2009 Vaccine 28: 965). Human monoclonal antibodies specific for each of TcdA and TcdB protect CDI patients from relapse (Lowy I et al. 2010 N Engl J Med 362: 197). Therefore, a vaccine that induces neutralizing antibodies against the toxins would be useful to prevent the disease or reduce its severity.

Protection against CDI is mediated through systemic and mucosal antibodies against the two toxins, although other virulence attributes are known to exist which may also contribute to manifestation of CDI (Aboudola, S. et al. 2003 Infection and immunity 71:1608-1610). Neutralizing monoclonal antibodies directed against TcdA inhibit fluid secretion in mouse intestinal loops and protect mice against systemic infection (Corthier, G. et al. 1991 Infect Immun 59:1192-1195). Co-administration of both anti-TcdA and anti-TcdB antibodies significantly reduces the mortality in a primary disease hamster model as well as in a less stringent relapse model (Babcock, G. J. et al. 2006 Infect Immun 74:6339-6347). Antibodies against C. difficile are present in the general population in individuals greater than two years of age, and a higher level of serum or mucosal antibody response is associated with less severe disease and less frequent relapse (Babcock, G. J. et al. 2006 Infect Immun 74:6339-6347; Kelly, C. P. et al. 1996 Antimicrob Agents Chemother 40:373-379; Kyne, L. et al. 2000 N Engl J Med 342:390-397). Humanized monoclonal antibodies against the toxins are under clinical trial for treatment of patients with CDAD. However, the mechanism by which serum antibodies prevent enterotoxicity and mucosal damage is not fully understood and antibodies are susceptible to degradation in the intestines and the efficacy is therefore compromised. Studies have shown that systemically administered human monoclonal IgG antibodies protect hamsters from acute C. difficile infection, but whether these antibodies can protect against chronic disease is unknown.

A toxoid vaccine has been generated by formaldehyde treatment and administered by intramuscular injection with alum as adjuvant (Kotloff, K. L. et al. 2001 Infect Immun 69:988-995; Sougioultzis, S. et al. 2005 Gastroenterology 128:764-770). Chemically detoxified toxoid induces a poorer mucosal response than molecules that target receptors on mucosal surfaces (Cropley, I. et al. 1995 Vaccine 13:1643-1648; Torres, J. F. et al. 1995 Infect Immun 63:4619-4627), since toxoid is unable to bind to the mucosal surface as a result of formaldehyde treatment (Kunkel, G. R. et al. 1981 Mol Cell Biochem 34:3-13). A vaccine that targets both TcdA and TcdB, and that elicits strong systemic and mucosal immunity to prevent CDI, reduces the severity, or eliminates an ongoing chronic disease is needed.

Compositions, methods and kits herein provide atoxic proteins for eliciting an immune response specific for a C. difficile toxin. The atoxic protein in various embodiments is a chimeric protein that includes at least one mutation that reduces the toxicity of the protein compared to a wildtype protein. The atoxic protein (e.g., Tcd138) in examples herein includes conservative sequence modifications to the nucleotide sequences or amino acid sequences described herein. As used herein, the term “conservative sequence modifications” refers to amino acid or nucleotide modifications that do not significantly affect or alter the characteristics of the atoxic protein, for example by substitution of an amino acid with a functionally similar amino acid. Such conservative modifications include substitutions, additions and deletions. Modifications of amino acid sequences or nucleotide sequences are achieved using any known technique in the art e.g., site-directed mutagenesis or PCR based mutagenesis. Such techniques are described in Sambrook et al., Molecular Cloning: A Laboratory Manual, fourth edition, Cold Spring Harbor Press, Plainview, N. Y., 2012 and Ausubel et al., Current Protocols in Molecular Biology, fifth edition, John Wiley & Sons, New York, N.Y., 2002.

Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine).

In certain embodiments, the amino acid sequence or nucleotide sequence of the atoxic protein is a sequence that is substantially identical to that of the wild type sequence of TcdA and TcdB. The term “substantially identical” is used herein to refer to a first sequence that contains a sufficient or minimum number of residues that are identical to aligned residues in a second sequence such that the first and second sequences can have a common structural domain and/or common functional activity. For example, amino acid sequences that contain a common structural domain having at least about 60% identity, or at least 70%, 75%, 80%, 85%, 90%, 95%, or 99% identity. For example, the atoxic protein has at least 60% identity, at least 65% identity, at least 70% identity, at least 75% identity, at least 80% identity, at least 85% identity, at least 90% identity, at least 95% identity, or at least 99% identity to the amino acid sequence of a wild-type bacterial sequence. In certain embodiments the nucleotide sequence of the atoxic protein has at least about 60%, 70%, 75%, 80%, 85%, 90%, 95%, or 99% identity to a wild-type bacterial toxin, e.g., TcdB gene.

Calculations of sequence identity between sequences are performed as follows. To determine the percent identity of two amino acid sequences for example, the sequences are aligned for optimal comparison purposes (e.g., gaps can be introduced in one or both of a first and a second amino acid sequence for optimal alignment). The amino acid residues at corresponding amino acid positions or nucleotide positions are then compared. When a position in the first sequence is occupied by the same amino acid residue or nucleotide as the corresponding position in the second sequence, then the proteins are identical at that position. The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences are accomplished using a mathematical algorithm. Percent identity between two amino acid sequences is determined using an alignment software program using the default parameters. Suitable programs include, for example, CLUSTAL W by Thompson et al., Nuc. Acids Research 22:4673, 1994, BL2SEQ by Tatusova and Madden, FEMS Microbiol. Lett. 174:247, 1999, SAGA by Notredame and Higgins, Nuc. Acids Research 24:1515, 1996, and DIALIGN by Morgenstern et al., Bioinformatics 14:290, 1998.

The immunogens are shown herein to be superior to toxoid or fragments thereof. The recombinant proteins in various embodiments mimic the native form with correct folding, and were observed to generate a full spectrum of neutralizing systemic and mucosal antibodies. Unlike chemically-detoxified toxoid or fragments that contain a small portion of TcdA, the atoxic holotoxins provided in various embodiments herein and carrying point mutations maintain the same adjuvant activity, antigenicity, and affinity to mucosal epithelium as do native toxins; thus induce greater protective immunity than toxoid and generate a wider spectrum of antibodies than fragments. Immunization with chimeric proteins was observed in Examples herein to induce potent protection in mice against lethal challenge with both TcdA and TcdB.

### Disease Manifestation and Therapeutic Approaches

CDI is acquired by the ingestion of vegetative organisms or spores, most likely the latter (Dubberke, E. R. et al. 2007 Am J Infect Control 35:315-318; Roberts, K. et al. 2008 BMC Infect Dis 8:7). Spores survive contact to gastric acidity and germinate in the gut. Antibiotic treatment is the most significant risk factor for the disease (Bartlett, J. G. 2006 Ann Intern Med 145:758-764). The clinical appearance of CDI is highly variable and ranges from asymptomatic to mild self-limiting diarrhea, to more severe pseudomembranous colitis. The most common symptom is diarrhea. Other common clinical symptoms include abdominal pain and cramping, increased temperature and leukocytosis. In mild cases of CDI, oral rehydration and withdrawal of antibiotics is often effective. More severe CDI cases are treated by oral administration of metronidazole or vancomycin.

This treatment however is associated with a relapse rate as high as 55% (Barbut, F. et al. 2000 J Clin Microbiol 38:2386-2388; Walters, B. A. et al. 1983 Gut 24:206-212), and the primary treatment option for recurrent CDI remains metronidazole or vancomycin. Other options, such as probiotics and anion-exchange resins, have limited efficacy and are potentially harmful (Gerding, D. N. et al. 2008 Clin Infect Dis 46 Suppl 1:S32-42). Experimental treatments in clinical development have included toxin-absorbing polymer, antibiotics, and toxin-specific human monoclonal antibodies (Anton, P. M. et al. 2004 Antimicrob Agents Chemother 48:3975-3979; Hinkson, P. L. et al. 2008 Antimicrob Agents Chemother 52:2190-2195; McVay, C. S. et al. 2000 Antimicrob Agents Chemother 44:2254-2258). A formaldehyde inactivated toxoid vaccine in clinical trial is administered intramuscularly (Kotloff, K. L. et al. 2001 Infect Immun 69:988-995; Sougioultzis, S. et al. 2005 Gastroenterology 128:764-770).

### Virulence Factors

CDI is primarily a toxin-mediated disease. Two extensively studied exotoxins, toxin A (TcdA) and toxin B (TcdB), are thought to be major virulence factors, and C. difficile strains that lack both toxin genes are non-pathogenic both for humans and animals (Elliott, B. et al. 2007 Intern Med J 37:561-568; Kelly, C. P. 1996 Eur J Gastroenterol Hepatol 8:1048-1053; Voth, D. E. et al. 2005 Clin Microbiol Rev 18:247-263). Purified TcdA possesses potent enterotoxic and pro-inflammatory activities, as determined in ligated intestinal loop studies in animals (Kurtz, C. B. et al. 2001 Antimicrobial agents and chemotherapy 45:2340-2347). TcdA is cytotoxic to cultured cells in nanogram quantities. TcdB has been reported to exhibit no enterotoxic activity in animals when administered as pure protein (Lyerly, D. M. et al. 1982 Infection and immunity 35:1147-1150; Lyerly, D. M. et al. 1985 Infect Immun 47:349-352). Isogenic strains that are deficient in each toxin demonstrated that TcdB is a key virulence factor in hamsters (Lyras, D., et al. 2009 Nature 458:1176-1179). Enterotoxic and pro-inflammatory activities of TcdB were observed from human intestinal xenografts in immunodeficient (SCID) mice (Savidge, T. C. et al. 2003 Gastroenterology 125:413-420). TcdA⁻B⁺ C. difficile strains are associated with pseudomembranous colitis in some patients (Shin, B. M. et al. 2007 Diagn Microbiol Infect Dis. 59:33-37). A small number of C. difficile isolates produce a binary toxin (CDT) that exhibits ADP-ribosyltransferase activity (Blossom, D. B. et al. 2007 Clin Infect Dis 45:222-227; Carter, G. P. et al. 2007 J Bacteriol 189:7290-7301; McMaster-Baxter, N. L. et al. 2007 Pharmacotherapy 27:1029-1039). The role of CDT in development of human disease is not well understood (Stare, B. G. et al. 2007 J Med Microbiol 56:329-335). In addition to toxins, several other factors may play roles in disease manifestation, including fimbriae and other molecules that facilitate adhesion, capsule production and hydrolytic enzyme secretion (Borriello, S. P. 1998 J Antimicrob Chemother 41 Suppl C:13-19). The surface layer proteins of C. difficile are involved in bacterial colonization, and antibodies specific for these proteins are partially protective (Calabi, E. et al. 2002 Infect Immun 70:5770-5778; O'Brien, J. B. et al. 2005 FEMS Microbiol Lett 246:199-205).

### Domains of TcdA and TcdB

TcdA (308 kD) and TcdB (269 kD) belong to a large clostridial cytotoxin (LCT) family and share 49% amino acid identity (Just, I. et al. 2004 Rev Physiol Biochem Pharmacol 152:23-47). The genes tcdA and tcdB and three accessory genes are located on the bacterial chromosome, forming a 19.6-kb pathogenicity locus (PaLoc) (142). TcdA and TcdB are structurally similar to each other (von Eichel-Streiber, C. et al. 1996 Trends Microbiol 4:375-382), consisting of at least three functional domains. The C-terminus contains a receptor binding domain (RBD), has a β-solenoid structure and is involved in receptor binding (Ho, J. G. et al. 2005 Proc Natl Acad Sci USA 102:18373-1837). The middle portion of the toxin primary structure is potentially involved in translocation of the toxin into target cells, and the N-terminus is a catalytic domain having glucosyltransferase activity (Hofmann, F. et al. 1997 J Biol Chem 272:11074-11078). The limits of the three domains have been defined in the literature (Giesemann, T. et al. 2008 J Med Microbiol 57:690-696). The GT domain was defined by expression of recombinant proteins deriving from DNA encoding the amino terminal (Hofmann, F. et al. 1997 J Biol Chem 272:11074-11078). In addition, the GT domain was recovered following cytosolic delivery and N-terminal amino acids determined (Pfeifer, G. et al. 2003 J Biol Chem 278:44535-44541), The crystal structure of RBD of TcdA revealed a solenoid-like structure. The boundary of the RBD in both toxins is near amino acid 1850. Interaction between the C-terminus and the host cell receptors is believed to initiate receptor-mediated endocytosis (Florin, I. et al. 1983 Biochim Biophys Acta 763:383-392; Karlsson, K. A. 1995 Curr Opin Struct Biol 5:622-635; Tucker, K. D. et al. 1991 Infect Immun 59:73-78). Although the intracellular mode of action remains unclear, it has been proposed that the toxins undergo a conformational change at the low pH of the endosomal compartment, leading to membrane insertion and channel formation (Giesemann, T. et al. 2006 J Biol Chem 281:10808-10815; Qa'Dan, M et al. 2000 Infect Immun 68:2470-2474). A host cofactor triggers a second structural change accompanied by autocatalytic cleavage and release of the catalytic domain into the cytosol (Pfeifer, G. et al. 2003 J Biol Chem 278:44535-44541; Reineke, J. et al. 2007 Nature 446:415-419). In the cytosol, the catalytic domain of toxins mono-O glucosylates low molecular mass GTPase of the Rho family, including Rho, Rac, and CDC42 (Just, I. et al. 1995 Nature 375:500-503). Glucosylation of Rho proteins inhibits the molecular switch function, blocking Rho GTPase-dependent signaling in intestinal epithelial cells, leading to alterations in the actin cytoskeleton, massive fluid secretion, acute inflammation and necrosis of the colonic mucosa (Just, I. et al. 1995 Nature 375:500-503; Pothoulakis, C. et al. 2001 Am J Physiol Gastrointest Liver Physiol 280:G178-183).

### Epidemiology and Diagnosis

The incidence of C. difficile in healthy adults is 3-5%, and as high as 60% in healthy neonates and infants (Larson, H. E. et al. 1982 J Infect Dis 146:727-733; Viscidi, R. et al. 1981 Gastroenterology 81:5-9). Despite the high carriage rate in neonates, symptomatic disease is uncommon (McFarland, L. V. et al. 2000 J Pediatr Gastroenterol Nutr 31:220-231). In adults with antibiotic usage and hospitalization, the rate of colonization increases substantially to 20-40% (Bartlett, J. G. 2006 Ann Intern Med 145:758-764). The standard test for infection is detection of C. difficile toxins in stool. Assays include cell culture-based cytotoxicity assay (Bartlett, J. G. et al. 1978 N Engl J Med 298:531-534) and enzyme immunoassays (EIAs; Russmann, H. et al. 2007 Eur J Clin Microbiol Infect Dis 26:115-119; Staneck, J. L. et al. 1996 J Clin Microbiol 34:2718-2721) to detect TcdA and/or TcdB in stool samples. Alternative detection methods include anaerobic culture of bacteria and detecting the bacterial antigen glutamate dehydrogenase (GDH).

### Pharmaceutical Compositions

An aspect of the present invention provides pharmaceutical compositions, wherein these compositions comprise an antigen from a toxin of C. difficile peptide or protein, and optionally further include an adjuvant, and optionally further include a pharmaceutically acceptable carrier. In various embodiments, the compositions include at least one atoxic protein or a source of expression of the protein, such that the protein illicits an immune response specific for a C. difficile toxin such as TcdA and TcdB.

In certain embodiments, these compositions optionally further comprise one or more additional therapeutic agents. In certain embodiments, the additional therapeutic agent or agents are selected from the group consisting of antibiotics particularly antibacterial compounds, anti-viral compounds, anti-fungals, and include one or more of growth factors, anti-inflammatory agents, vasopressor agents, collagenase inhibitors, topical steroids, matrix metalloproteinase inhibitors, ascorbates, angiotensin II, angiotensin III, calreticulin, tetracyclines, fibronectin, collagen, thrombospondin, transforming growth factors (TGF), keratinocyte growth factor (KGF), fibroblast growth factor (FGF), insulin-like growth factors (IGF), epidermal growth factor (EGF), platelet derived growth factor (PDGF), neu differentiation factor (NDF), hepatocyte growth factor (HGF), and hyaluronic acid.

As used herein, the term “pharmaceutically acceptable carrier” includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences Ed. by Gennaro, Mack Publishing, Easton, Pa., 1995 discloses various carriers used in formulating pharmaceutical compositions and known techniques for the preparation thereof. Carriers are selected to prolong dwell time for example following any route of administration, including IP, IV, subcutaneous, mucosal, sublingual, inhalation or other form of intranasal administration, or other route of administration.

Some examples of materials that can serve as pharmaceutically acceptable carriers include, but are not limited to, sugars such as glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil, safflower oil, sesame oil, olive oil, corn oil, and soybean oil; glycols such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

In yet another aspect, according to the methods of treatment of the present invention, the immunization is promoted by contacting the subject with a pharmaceutical composition, as described herein. Thus, the invention provides methods for immunization comprising administering a therapeutically effective amount of a pharmaceutical composition comprising active agents that include an immunogenic toxin protein of C. difficile having an associated antigenic determinant for at least one of TcdA and TcdB, to a subject in need thereof, in such amounts and for such time as is necessary to achieve the desired result. It will be appreciated that this encompasses administering an inventive vaccine as described herein, as a preventive or therapeutic measure to promote immunity to infection by C. difficile, to minimize complications associated with the slow development of immunity (especially in compromised patients such as those who are nutritionally challenged, or at risk patients such as the elderly or infants).

In certain embodiments of the present invention a “therapeutically effective amount” of the pharmaceutical composition is that amount effective for promoting appearance of antibodies in serum specific for the toxins of C. difficile, or disappearance of disease symptoms, such as amount of antigen or toxin or bacterial cells in feces or in bodily fluids or in other secreted products. The compositions, according to the method of the present invention, may be administered using any amount and any route of administration effective for generating an antibody response. Thus, the expression “amount effective for promoting immunity”, as used herein, refers to a sufficient amount of composition to result in antibody production or remediation of a disease symptom characteristic of infection by C. difficile.

The exact dosage is chosen by the individual physician in view of the patient to be treated. Dosage and administration are adjusted to provide sufficient levels of the active agent(s) or to maintain the desired effect. Additional factors which may be taken into account include the severity of the disease state; contact to infectious agent in the past or potential future contact; age, weight and gender of the patient; diet, time and frequency of administration; drug combinations; reaction sensitivities; and tolerance/response to therapy. Long acting pharmaceutical compositions might be administered every three to four days, every week, or once every two weeks depending on half-life and clearance rate of the particular composition.

The active agents of the invention are preferably formulated in dosage unit form for ease of administration and uniformity of dosage. The expression “dosage unit form” as used herein refers to a physically discrete unit of active agent appropriate for one dose to be administered to the patient to be treated. It will be understood, however, that the total daily usage of the compositions of the present invention will be decided by the attending physician within the scope of sound medical judgment. For any active agent, the therapeutically effective dose can be estimated initially either in cell culture assays or in animal models, usually mice, rabbits, dogs, or pigs or piglets or other suitable animals. The animal models described herein including that of chronic or recurring infection by C. difficile is also used to achieve a desirable concentration range and route of administration. Such information can then be used to determine useful doses and routes for administration in humans.

A therapeutically effective dose refers to that amount of active agent which ameliorates at least one symptom or condition. Therapeutic efficacy and toxicity of active agents can be determined by standard pharmaceutical procedures in cell cultures or experimental animals, e.g., ED50 (the dose is therapeutically effective in 50% of the population) and LD50 (the dose is lethal to 50% of the population). The dose ratio of toxic to therapeutic effects is the therapeutic index, and it can be expressed as the ratio, LD50/ED50. Pharmaceutical compositions which exhibit large therapeutic indices are preferred. The data obtained from cell culture assays and from animal studies are used in formulating a range of dosage for human use.

The therapeutic dose shown in examples herein is at least about 1 μg per kg, at least about 5, 10, 50, 100, 500 μg per kg, at least about 1 mg/kg, 5, 10, 50 or 100 mg/kg body weight of the purified toxin vaccine per body weight of the subject, although the doses may be more or less depending on age, health status, history of prior infection, and immune status of the subject as would be known by one of skill in the art of immunization. Doses may be divided or unitary per day and may be administered once or repeated at appropriate intervals.

### Administration of Pharmaceutical Compositions

After formulation with an appropriate pharmaceutically acceptable carrier in a desired dosage, the pharmaceutical compositions of this invention can be administered to humans and other mammals topically (as by powders, ointments, or drops), orally, rectally, mucosally, sublingually, parenterally, intracisternally, intravaginally, intraperitoneally, bucally, sublingually, ocularly, or intranasally, depending on preventive or therapeutic objectives and the severity and nature of a pre-existing infection.

In various embodiments of the invention herein, it was observed that high titers of antibodies, sufficient for protection against a lethal dose of C. difficile toxin, were produced after administration of the engineered atoxic toxin proteins provided herein. Liquid dosage forms for oral administration include, but are not limited to, pharmaceutically acceptable emulsions, microemulsions, solutions, suspensions, syrups and elixirs. In addition to the active agent(s), the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Dosage forms for topical or transdermal administration of an inventive pharmaceutical composition include ointments, pastes, creams, lotions, gels, powders, solutions, sprays, inhalants, or patches. The active agent is admixed under sterile conditions with a pharmaceutically acceptable carrier and any needed preservatives or buffers as may be required. Administration may be therapeutic or it may be prophylactic.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized prior to addition of spores, for example, by filtration through a bacterial-retaining filter, or by incorporating sterilizing agents in the form of sterile solid compositions which can be dissolved or dispersed in sterile water or other sterile injectable medium prior to use. In order to prolong the effect of an active agent, it is often desirable to slow the absorption of the agent from subcutaneous or intramuscular injection. Delayed absorption of a parenterally administered active agent may be accomplished by dissolving or suspending the agent in an oil vehicle. Injectable depot forms are made by forming microencapsule matrices of the agent in biodegradable polymers such as polylactide-polyglycolide. Depending upon the ratio of active agent to polymer and the nature of the particular polymer employed, the rate of active agent release can be controlled. Examples of other biodegradable polymers include poly(orthoesters) and poly(anhydrides). Depot injectable formulations are also prepared by entrapping the agent in liposomes or microemulsions which are compatible with body tissues.

Compositions for rectal or vaginal administration are preferably suppositories which can be prepared by mixing the active agent(s) of this invention with suitable non-irritating excipients or carriers such as cocoa butter, polyethylene glycol or a suppository wax which are solid at ambient temperature but liquid at body temperature and therefore melt in the rectum or vaginal cavity and release the active agent(s).

Solid dosage forms for oral, mucosal or sublingual administration include capsules, tablets, pills, powders, and granules. In such solid dosage forms, the active agent is mixed with at least one inert, pharmaceutically acceptable excipient or carrier such as sodium citrate or dicalcium phosphate and/or a) fillers or extenders such as starches, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof.

Solid compositions of a similar type may also be employed as fillers in soft and hard-filled gelatin capsules using such excipients as milk sugar as well as high molecular weight polyethylene glycols and the like. The solid dosage forms of tablets, dragees, capsules, pills, and granules can be prepared with coatings and shells such as enteric coatings, release controlling coatings and other coatings well known in the pharmaceutical formulating art. In such solid dosage forms the active agent(s) may be admixed with at least one inert diluent such as sucrose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active agent(s) only, or preferentially, in a certain part of the intestinal tract, optionally, in a delayed manner. Examples of embedding compositions which can be used include polymeric substances and waxes.

### Identifying Routes of Immunization

Routes suitable for inducing systemic and mucosal antibodies and protective responses are identified by comparing oral, intranasal (IN), or sublingual (SL) immunization regimens, with a view to establish systemic protection levels similar or superior to IP immunization as well as mucosal protection.

The various embodiments of the composition, the atoxic protein contains intact receptor binding domain(s) of native toxins, and is likely have similar affinities for epithelial cells as do wild type toxins. Consequently, mucosal immunity is induced by contact of mucosal surfaces (oral, IN, or SL) to these immunogens. Several routes of mucosal immunizations are compared and the induction of systemic and mucosal antibody responses was assessed. The usage of mucosal adjuvants is also evaluated.

Beginning with the optimal dose established as described in Examples herein, groups of mice are immunized multiple times with atoxic proteins (e.g., Tcd138), IN or SL (with or without mucosal adjuvant), or orally (encapsulated). Serum and fecal antibody responses are measured after each immunization. One week after the last immunization mice ware challenged IP with the corresponding LD₅₀ᵢ toxin established, and the protective responses to systemic challenge are compared with groups immunized IP and with a placebo group. Systemic antibodies in serum and secretory IgA and IgG against toxins in feces and gut contents are measured and neutralizing titers for blocking cytotoxicity in cell culture are determined. Mucosal protection is evaluated in the ligated ileal loops of immunized mice directly injected with toxin. Dose optimization of the immunogen(s), combined with alternate mucosal adjuvants, follows if the levels of antibody and protective responses are considerably less than that accomplished by parenteral immunization, and/or if the mucosal antibody and protective responses are considered to be low. These assays establish whether mucosal immunization is efficient in terms of antibody and protective responses as IP immunization, and whether a protective mucosal immunity is induced by mucosal immunization.

### Adjuvants

Intraperitoneal (IP) immunization with atoxic protein using alum as adjuvant was shown to induce strong IgG response and systemic protection. Importantly, alum is an FDA approved adjuvant for human vaccination. Therefore, parenteral immunizations included alum as adjuvant, including the placebo.

The bacterial enterotoxins cholera toxin (CT) from Vibrio cholerae and the heat labile toxin (LT) from E. coli are the most commonly used mucosal adjuvants, boosting immune responses to unrelated antigens co-administered by oral or nasal routes (Rappuoli, R. et al. 1999 Immunol Today 20:493-500). However, the wild types of these enteric toxins are toxic, therefore, extensive studies have been carried to reduce the toxicity of CT and LT while retain their adjuvant activities (Pizza, M. et al. 2001 Vaccine 19:2534-2541). An example is the mutant LT (mLT) which carries a mutation in the proteolytic site of the A subunit at amino acid 192 that abrogates cleavage and attenuates the toxicity of the protein (Dickinson, B. L. et al. 1995 Infect Immun 63:1617-1623; Cavalcante, I. C. et al. 2006 Infect Immun 74:2606-2612). In various embodiments, the atoxic proteins described herein possesses adjuvant activity as strong as CT or LT after intranasal administration. In addition, toxins TcdA and TcdB have high affinity to epithelial cells, thus an optimal dose of the atoxic immunogens described herein, without extraneous adjuvant, may be sufficient to induce strong neutralizing IgG and IgA responses.

Comparisons were made between groups of animals immunized with immunogens lacking adjuvant, or including mutant LT (mLT) or mutant CpG. mLT has been used in animal and in human studies (Dickinson, B. L. et al. 1995 Infect Immun 63:1617-1623; Uddowla, S. et al. 2007 Vaccine 25:7984-7993). mLT was constructed using site-directed mutagenesis to create a single amino acid substitution within the disulfides subtended region of the A subunit separating A1 from A2 (Dubberke, E. R. et al. 2007 Am J Infect Control 35:315-318). This single amino acid change altered the proteolytically sensitive site within this region, rendering the mutant insensitive to trypsin activation. The outcome of immunization of each immunogen mixed with 5 μg or 10 μg of mLT was compared and results are presented in the Examples herein.

The immunomodulatory properties of CpGs (Kindrachuk, J. et al. 2009 Vaccine 27:4662-4671) used herein include those useful for a number of potential medical applications: priming the innate immune response, as anti-allergens, for the treatment of a variety of malignancies, and as adjuvants for improving vaccination efficiency, especially in individuals with poor immune responses. Indeed these molecules have been demonstrated to enhance human, murine, and porcine neonatal immune responses, although the use of CpGs in adjuvant formulations was previously demonstrated to skew vaccine-induced immune responses towards a Th1-bias (Garlapati, S. et al. 2009 Vet Immunol Immunopathol 128:184-191). In the context of a vaccine adjuvant, a balanced Th1/Th2 response is desirable since the modulation of Th1 and Th2 contributions influences the balance between protection and immunopathology (Singh, V. K. et al. 1999 Immunol Res 20:147-161).

### Intranasal Immunization (IN)

The mucosal nasal route of immunization induces an immune response resulting in systemic and/or mucosal antibody response in mice, and in the intestines in humans (Kozlowski, P. A. et al. 2002 J Immunol 169:566-574; Rudin, A. et al. 1999 Infect Immun 67:2884-2890). The nasal route avoids protein digestion and degradation in the GI tract, allowing far less antigen to be delivered than the oral route (Kozlowski, P. A. et al. 2002 J Immunol 169:566-574). Therefore, the nasal route of immunization is considered herein to have a great potential (Neutra, M. R. et al. 2006 Nat Rev Immunol 6:148-158).

For intranasal route of immunization, an atoxic protein such as Tcd138 with or without adjuvant is delivered into each nostril. A suitable volume of atoxic protein per nostril ensures that the immunogens is distributed inside of nasal cavity. Higher volumes, such as 30 μl, may lead to nasal/pulmonary immunization (Southam, D. S. et al. 2002 Am J Physiol Lung Cell Mol Physiol 282:L833-839). Binding of the immunogens to nasal epithelium is evaluated. The use of LT as adjuvant alters antigen trafficking in the nasal tract. This is the case with wild type LT but not mLT, since this adjuvant-dependent redirection of antigen is dependent on ADP-ribosyltransferase activity (van Ginkel, F. W. et al. 2005 Infect Immun 73:6892-6902).

### Sublingual Immunization (SL)

The SL route has been used for many years to deliver low molecular weight drugs to the bloodstream (Zhang, H. et al. 2002 Clin Pharmacokinet 41:661-680) and for immunotherapy directed towards allergens (O'Hehir, R. E. et al. 2007 Curr Med Chem 14:2235-2244). This route of vaccination has a potential for ease of delivery and for inducing broad systemic and mucosal immune response (Cuburu, N. et al. 2007 Vaccine 25:8598-8610). SL immunization induces intestinal mucosal immunity against infection with enteric pathogens (Huang, C. F. et al. 2008 J Pediatr Gastroenterol Nutr 46:262-271).

The sublingual mucosa encompasses the ventral side of the tongue and the floor of the mouth. For SL immunization in various examples, mice are anesthetized with ketamine/xylazine, and 5 μl of an atoxic protein with or without adjuvant is delivered at the ventral side of the tongue and directed toward the floor of the mouth. Animals are maintained with heads placed in anteflexion for 30 minutes.

### Oral Immunization

Direct stimulation of the gut mucosa induces effective protection against enteric infections. Attempts to deliver inactivated or subunit vaccines of particles, proteins or DNA have been tried by many groups with mixed results. Oral vaccination is safes and effective method for protecting the gut against infection. It is also treacherous route because of proteolytic or hydrolyzing digestive enzymes, bile salts, and extreme pH as well as rapid movement of contents and often limited access to the mucosal wall.

PLG polymers were selected for use in Examples herein because the polymers used for encapsulation are non-immunogenic and have a known record of safety. This has been shown in their use for other purposes, such as in drug delivery and in surgical suture materials. Poly (lactide-co-glycolide) is hydrolyzed in vivo to two naturally occurring substances, lactic acid and glycolic acid.

### Uses of Pharmaceutical Compositions

As discussed above and described in greater detail in the Examples, engineered toxin proteins are provided herein that are effective in eliciting antibody production for toxins of C. difficile and for preventing disease symptoms, infection, and death. In general, it is believed that these vaccines will be clinically useful in immunizing subjects for resistance to CDI. The vaccines herein are particularly useful to treat compromised patients, particularly those anticipating therapy involving, for example, immunosuppression and complications associated with systemic treatment with steroids, radiation therapy, non-steroidal anti-inflammatory drugs (NSAID), anti-neoplastic drugs and anti-metabolites. Patients receiving large routine doses of antibiotic therapy which is known to eliminate or reduce intestinal flora, for example surgical patients and those experiencing trauma such as arising from accidents or battle field wounds, are populations that can be immunized to prevent development of CDI as C. difficile flourishes absent competing normal bacterial flora. It is envisioned also that the vaccines herein may be used prophylactically to immunize entire populations such as school age children or members of the military for prevention of CDI, particularly after catastrophes such as earthquakes and floods.

### Systemic and Mucosal Antibodies in Protection Against CDI

Both systemic and mucosal immunity provide protection against enteric pathogens and pathogenic products, such as toxins (Huang, C. F. et al. 2008 J Pediatr Gastroenterol Nutr 46:262-271; Perez, J. L. et al. 2009 Vaccine 27:205-212). Because TcdA and TcdB are essential virulence factors for C. difficile, an antitoxin preparation can convey full protection from oral C. difficile challenge in animals (Kink, J. A. et al. 1998 Infect Immun 66:2018-2025; Lyerly, D. M. et al. 1991 Infect Immun 59:2215-2218). Antibodies against both toxins, but not against TcdA or TcdB alone, protect against toxigenic C. difficile infection in a hamster model (Fernie, D. S. et al. 1983 Dev Biol Stand 53:325-332; Kim, P. H. et al. 1987 Infect Immun 55:2984-2992; Libby, J. M. et al. 1982 Infect Immun 36:822-829). An evaluation of the routes of delivery of toxoid vaccine in hamsters assessing protection from both lethal disease and diarrhea have found that a combination of mucosal and parental immunization provided complete protection from diarrhea and death, showing that induction of both systemic and mucosal immunity was necessary for optimal protection (Torres, J. F. et al. 1995 Infect Immun 63:4619-4627). The systemic administrated human monoclonal IgG antibodies protected hamsters from acute CDI and mortality (Babcock, G. J. et al. 2006 Infect Immun 74:6339-6347). Whether these antibodies can protect against chronic diseases is unknown. Since these are administered systemically and are passively acquired antibodies, the duration of protection is limited and costly.

In humans, a high level of antitoxin antibodies in serum is associated with less severe disease and less frequent relapses (Kyne, L. et al. 2000 N Engl J Med 342:390-397). Following symptomatic infection, most individuals develop antibodies against the two toxins in serum (Aronsson, B. et al. 1985 Infection 13:97-101; Viscidi, R. et al. 1983 J Infect Dis 148:93-100), including toxin-neutralizing IgA in serum and stool (Johnson. S. et al. 1995 Infect Immun 63:3166-3173). Systemic and mucosal antibody response appears to be associated with protection from subsequent infections. Disease progression and recurrence seem to be associated with the different subsets of antibodies in the circulation (Katchar, K. et al. 2007 Clin Gastroenterol Hepatol 5:707-713), and the exact reason behind this observation is unclear. A TcdA-specific antibody substantially enhanced the cytotoxic activity of TcdA on macrophages or monocytes through Fe gamma receptor I-mediated endocytosis as was shown in He, X. et al. 2009 Infect Immune 77:2294-2303, which is incorporated herein by reference hereby in its entirety.

### Vaccine Development

Antibodies specific for both toxins are needed to protect animals colonized with highly toxigenic strains (Babcock, G. J. et al. 2006 Infect Immun 74:6339-6347). The toxicity of unmodified C. difficile toxins prevents direct use as vaccines; therefore, toxoid generated by formaldehyde crosslinking or toxin fragments that lack the catalytic domain have been utilized as candidate vaccines (Ghose, C. et al. 2007 Infect Immun 75:2826-2832; Torres, J. F. et al. 1995 Infect Immun 63:4619-4627; Ward, S. J. et al. 1999 Infect Immun 67:5124-5132).

Parental toxoid immunization provides only partial protection against CDI in the acute hamster disease model and induces serum IgG responses in human volunteers (Kotloff, K. L. et al. 2001 Infect Immun 69:988-995; Torres, J. F. et al. 1995 Infect Immun 63:4619-4627). It is however unclear whether this regimen of vaccination is effective against chronic disease and provides mucosa(protection. Due to the nature of the intestinal infection, a mucosal route of vaccination capable of generating systemic and mucosal antibody responses against C. difficile toxins is preferable to other routes of vaccine administrations. Consequently, mucosal routes of immunization have been tested using toxoids with the mucosal adjuvant cholera toxins (CT). A combination of intranasal and intraperitoneal immunization provided full protection from both lethal disease and diarrhea in hamsters after C. difficile oral challenge (Torres, J. F. et al. 1995 Infect Immun 63:4619-4627). Transcutaneous routes of toxoid immunization caused a mucosal IgA response with CT as adjuvant (Ghose, C. et al. 2007 Infect Immun 75:2826-2832). Chemically detoxified toxoid induces a poorer mucosal response than molecules that can target receptors on mucosal surfaces (Cropley, I. et al. 1995 Vaccine 13:1643-1648; Torres, J. F. et al. 1995 Infect Immun 63:4619-4627), since toxoid is unable to bind to the mucosal surface due to the nature of formaldehyde treatment (Kunkel, G. R. et al. 1981 Mol Cell Biochem 34:3-13). As such, strong mucosal adjuvants, such as CT or E. coli heat liable toxin (LT), are necessary for induction of mucosal immunity.

Another form of experimental vaccine for CDI is recombinant expressed toxin fragments that are devoid of GT domain and are therefore non-toxic. Although TcdB may be more important than TcdA in pathogenesis of the disease in CDI (Lyras, D., et al. 2009 Nature 458:1176-1179), only fragments that contain a portion of receptor binding domain of TcdA have been tested as candidate vaccine (Sauerborn, M. et al. 1997 FEMS Microbiol Lett 155:45-54; Ward, S. J. et al. 1999 Infect Immun 67:5124-5132). Recombinantly expressed toxin fragments are relatively easy to produce in large quantities. Deletion of significant parts of the holotoxin may affect the overall receptor binding and uptake of toxin fragments by the epithelium. In addition, it is possible that the deletion of a large portion of the toxins affects stereo composition of the fragment. Consequently, fragments have lost the ability to induce antibodies against the deleted portion and against stereotypically significant epitopes of the holotoxins, reducing considerably their antigenicity. Holotoxins in contrast induce antibodies specific for epitopes across the entire toxins (Babcock, G. J. et al. 2006 Infect Immun 74:6339-6347).

Intramuscular immunization with a DNA vector expressing C-terminal receptor-binding domain of TcdA was shown to induce systemic IgG response against that fragment, and the immunized mice survived from a challenge with wild type TcdA (Gardiner, D. F. et al. 2009. Vaccine 27:3598-3604). DNA vaccines have been shown to generate mucosal immune response (van Ginkel, F. W. et al. 2000 Emerg Infect Dis 6:123-132). Plasmid DNA was used in clinical trials to induce systemic antibodies and CTL against several pathogens, including hepatitis B virus, herpes simplex virus, HIV, malaria, and influenza, but failed to induce adequate responses in the mucosal compartment in these cases (van Ginkel, F. W. et al. 2000 Emerg Infect Dis 6:123-132). Both holotoxins have large sizes and are consequently predicted to have over 20 0- and N-glycosylation sites as expressed in mammalian cells rendering difficulty in expressing whole or even a large portion of the toxin genes in mammalian cells without significant alteration of the stereo composition of the protein by glycosylation. Small portions of toxin fragment expressed using a DNA vectore were observed to have reduced antigenicity and therefore less valuable as vaccines.

Because options for preventing and treating CDI are rapidly diminishing, particularly against recently emerged hypervirulent C. difficile strains, novel strategies are needed. Current vaccines using toxoid, toxin fragments, or fragment-expressing DNA vectors have various disadvantages discussed above. Prior attempts to express C. difficile holotoxins have been limited (Park, E. J. et al. 1999 Exp Mol Med 31:101-107; Pizza, M. et al. 1994 J Exp Med 180:2147-2153).

These problems are addressed in Examples herein, in which wild type and GT-deficient holotoxin proteins were expressed in an endotoxin-free B. megaterium system with a high expression yields. In various embodiments the atoxic proteins have intact C-terminal regions and conformations, and to maintain equivalent adjuvant activity, antigenicity, and affinity to the mucosal epithelium as wild type toxins. Immunization of mice with atoxic holotoxin proteins is here observed to have induced stronger antibody response and protective immunity than did the corresponding toxoid, and induced a wider spectrum antibody response then did toxin fragment. In addition, vaccination of mice with a specifically designed chimeric protein containing elements from both TcdA and TcdB induced antibodies specific for both toxins and protected mice from lethal challenge by both toxins. Therefore, atoxic toxin proteins such as Tcd138, derivates and homologs thereof in Examples herein are evaluated herein as vaccine candidates for safety, immunogenicity, and assessment of efficacy as administered by various routes and regimens of immunizations.

Immunogens were constructed and were evaluated for relative efficacy to induce robust mucosal and/or systemic protection against oral challenge by C. difficile. Several regimens of mucosal immunizations (oral, intranasal and sublingual) designed to induce protection against systemic and mucosal challenge wild type toxins were assessed for efficacy. Mucosal immunization is suitable to administer to patients who would benefit from receiving multiple boosters. The protective efficacies of the various immunization regimens analyzed herein were assessed using a mouse acute infection model. Immunization methods evaluated as efficacious by data obtained using the mouse infection studies were then evaluated in the chronic gnotobiotic piglet model of CDI. Orally infected piglets display several key characteristics observed in humans with CDI. Depending on age and infectious dose, these include symptoms of acute illness of diarrhea, anorexia and possible fatality; or chronic disease with typical pseudomembranous colitis, inflammation and profound mucosal damage, manifested with prolong intermittent diarrhea, poor health, and weight loss.

Safety and immunogenicity of immunogens were observed herein, indicating that the atoxic proteins described herein would be suitable for development of an effective needle-free, temperature resistant vaccine candidate which simultaneously would protect patients against gastrointestinal and systemic manifestations of illness. Mucosal adjuvants such as mLT and CpG for intranasal and sublingual immunizations, and CT (modified cholera toxin) microencapsulation for oral immunization were examined.

### Animal Disease Models

CDI has been studied in a number of animal species, including hamsters, guinea pigs, rabbits, and germ-free mice and rats (Abrams, G. D. et al. 1980 Gut 21:493-499; Czuprynski, C. J. et al. 1983 Infect Immun 39:1368-1376; Fekety, R. et al. 1979 Rev Infect Dis 1:386-397; Knoop, F. C. 1979 Infect Immun 23:31-33). The most widely used model is the hamster, in which CDI is induced with toxigenic C. difficile infection of antibiotic treated animals. The disease in hamsters primarily affects the cecum with some involvement of the ileum; animals develop diarrhea which is fatal due to severe enterocolitis. The lethal disease in hamsters does not represent the usual course and spectrum of CDI in humans. The hamster model has been used for three decades to study therapy and mechanisms of disease. Animal models that more closely resemble human CDI have been developed (Chen, X. et al. 2008 Gastroenterology 135:1984-1992; 129), including a C57BL/6 mouse model which is susceptible to C. difficile after contact to a mixture of antibiotics for three days (Chen, X. et al. 2008 Gastroenterology 135:1984-1992). The mice developed diarrhea and lost weight. Disease severity varied from fulminant to minimal in accordance with the challenge dose. Typical histologic features of CDI were evident.

C. difficile also causes naturally occurring diarrhea-associated disease in swine, most typically during the first seven days of life (Songer, J. G. et al. 2006 Anaerobe 12:1-4; Songer, J. G. et al. 2000 Swine Health and Production 8:185-189; Songer, J. G. et al. 2005 J Vet Diagnost Investigat 17:528-536). CDI is the most common diagnosis of enteritis in neonatal pigs (Songer, J. G. et al. 2006 Anaerobe 12:1-4), perhaps, because of the similarities in the anatomy and physiology of the digestive track, nature of the diet, and the associated gut microflora which result from such combination of factors. This makes piglets a potential model for CDI. The germfree or the gnotobiotic (GB) piglet offers additional advantage in that it is a well characterized, controlled, optimized and standardized model which requires no antibiotic treatment to sterilize the gut. Challenging piglets with the hypervirulent strain 027/BI/NAP1 produced consistent results, with 100% colonization within 48 hours of inoculation, 100% morbidity, and severity of disease and mortality dependent upon dose and age at inoculation (Steele, J. et al. 2010 J Infect Diseases 201:428). Additionally, the piglet model offers a range of disease spectrum, from acute and lethal to chronic diarrhea with the characteristic pseudomembranous colitis with intensity and duration that can readily be manipulated under a controlled laboratory setting. The range of clinical signs, including systemic consequences, is similar to that observed in human cases, making the GB piglet an attractive model to perform preclinical evaluation of vaccine candidates and therapeutic agents.

### Use of Animal Models to Evaluate Efficacy of Immunogens

Immunogens are evaluated for efficacy in preventing the development of symptoms of diarrhea and/or systemic intoxication consistent with acute or chronic CDI, using the gnotobiotic piglet model of C. difficile infection, and the mouse CDI model.

There are no in vitro techniques available that can mimic an in vivo immune response and pathologic outcome generated as a consequence of toxin inoculation. Murine models of infection are useful models for analyzing naturally occurring host immune responses due to ease of manipulation, existence of genetically inbred strains and abundant immunological reagents. A systemic challenge model is used to evaluate the protection generated by parenteral or mucosal immunizations. The ileal loop assays include precise control of the dose of toxin inoculated into a microenvironment, and therefore dissection and definition of mucosal protection generated by a mucosal route of immunization. Traditionally, the hamster is a widely used animal model of CDI. This model has been used extensively for CDI studies, and hamsters are extremely sensitive to the infection. Mortality often approaches 100% within 48 hours of infection with virulent strains, and a fatal disease may develop from just one colony forming unit cfu (Keel, M. K. et al. 2006 Vet Pathol 43:225-240). Acute onset in hamsters leaves little time for investigation of events in pathogenesis compared to disease in humans. For these reasons, investigators actively seek animal model that closely resembles human causes of the diseases. The mouse infection model closely resembles human course of the disease (Chen, X. et al. 2008 Gastroenterology 135:1984-1992). Use of the two animal models allows evaluation of safety of candidate vaccines essential prior to application to control or treatment of CDI in humans. Both the mouse and the piglet CDI models described herein can be used to generate preclinical vaccine evaluation data required for PDP leading to Phase I clinical trials in human volunteers.

The efficacy of the top ranked regimens of systemic and/or mucosal immunizations is assessed for protection of animals against acute and chronic CDI induced by orally challenged animal models with C. difficile. Considering the complex of CDI manifestations (from mild diarrhea, pseudomembranous colitis, to fulminant disease and recurrence and multiple relapses), animal CDI models are needed to fully evaluate the efficacy of immunogens with a view to perform preclinical evaluation on one of them as a potential candidate vaccine. Both mouse and gnotobiotic (GB) piglet models are used to evaluate whether an immunization, based on the above findings, is capable of preventing animals orally challenged with C. difficile from developing CDI. In addition, assessment of vaccination reduction or elimination of C. difficile-mediated, ongoing, chronic diseases in the piglet infection model is performed. The persistence and magnitude of antitoxin antibodies are measured over several months and assessment is made of necessity of at least one additional booster to prevent recurrence or relapse of CDI in piglets.

Efficacy of the top ranked regimens of systemic and/or mucosal immunizations is initially examined for ability to protect mice against acute CDI induced by oral challenge with C. difficile followed by examining the efficacy of selected regimens in the piglet model of chronic CDI.

In a piglet model, groups of piglets are maintained for additional three months after the oral challenge with C. difficile, to monitor the levels of specific serum and secretory antibody, after which they are challenged with C. difficile a second time to assess protection against recurrence/relapse; if the specific antibody levels are deemed low, a booster immunization is given before the second challenge. The efficacy of such vaccines is evaluated using symptomatic, pathological and immunological parameters established for the piglet model. Comprehensive preclinical evaluation of efficacy of candidates is performed using atoxic proteins such as Tcd138, using various routes with or without adjuvant, as the basis for a vaccine. The best candidate vaccine is selected for clinical evaluation. The examples herein show data obtained describing duration of protection against relapses, and the benefit of booster immunizations.

### EXAMPLES

### Example 1

### Treatment Design for Mice

Absence of toxicity of the mutant holotoxins and cTxAB are determined by assay of cytotoxicity and in vivo toxicity in mice challenged systemically.

Immunizations are by intraperitoneal (IP) injection of 5 or 10 μg of purified antigens with alum as adjuvant. Antibody titers are measured by standard ELISA. Serum neutralizing titers are measured by blocking cytotoxicity of wild type toxins on mouse intestinal epithelial line CT26 cells. To evaluate the protection of vaccination against systemic toxins, the immunized mice are challenged IP with lethal doses of either wild type TcdA or TcdB, and mouse disease and mortality are monitored.

To evaluate the protective immunity against CDI, immunized mice are orally challenged with C. difficile vegetative cells or spores and development of symptoms such as diarrhea, weight loss, and mouse survival is monitored. Intestinal inflammation and tissue damage is assessed by histopathology analysis.

To evaluate the protection by atoxin protein vaccination for recurrent CDI, immunized mice are treated with antibiotic cocktail and then orally rechallenged with C. difficile spores 30-day after initial C. difficile challenge. Mouse survival and symptoms of the disease are monitored.

Mice are treated in groups of 10 C57BL/6 or Balb/c mice, aged 6-8 weeks. Each set of treatments includes a positive control (toxoid) and a negative control (vehicle plus adjuvant where appropriate). Animals are vaccinated three times at two weekly intervals, and one week after the last immunization mice are further challenged with the relevant wild type toxin. Mice are treated with antibiotics after the last immunization and before oral challenge with laboratory strain VPI 10463, the hypervirulent C. difficile strain 027, or with the control (tcdA⁻ tcdB⁻ avirulent) strain CD37. Before each immunization or challenge with toxin or bacteria, sera and fecal samples are collected and analyzed for specific antibody isotypes by ELISA and by the cell cytotoxicity assay. After challenge with toxin or bacteria mice are monitored closely for symptoms of illness which include reluctance to move, anorexia, arched back, lethargy, loss of body weight, pasty stools, ruffled coat, and recumbency. Seriously sick animals are euthanized. In animals challenged orally with C. difficile, bacterial excretion in feces is quantified, and visceral organs are formalin-fixed and examined histologically for abnormalities as shown in Examples herein. The presence of toxins circulating in blood (animals challenged with toxins), or in blood and in feces (animals challenged orally with C. difficile), is quantified, using the ultrasensitive assay as described (He, X. et al. 2009 J Microbiol Methods 78:97-100, incorporated herein by reference in its entirety).

Balb/c or C57BL/c mice were treated with antibiotic cocktail (a mixture of kanamycin, gentamicin, colistin, metronidazole, and vancomycin) followed with oral inoculation of C. difficile as described previously (Chen, X et al. 2008 Gastroenterology 135: 1984). Ten days after the third immunization, mice were given 10⁵ CFU of vegetative bacteria (laboratory VPI10463 strain) using gavage. To assess long-term immunity, mice were orally challenged with 10⁶ CFU of vegetative bacteria three months after the third immunization. In some Examples, the immunized mice were challenged with 10⁶ spores of UK1 (027/B1/NAP1 strain, VA Chicago Health Care System). To induce relapse CDI, surviving mice were given antibiotic cocktail treatment followed with an oral C. difficile spore (10⁶/mouse) inoculation 30 day-post the primary infection. The secondary challenge induces a similar clinic manifestation and intestinal histopathology as the primary CDI. The recurrent disease and death were monitored.

### Example 2

### Toxin Shedding after C. difficile Challenge

After primary and secondary challenge with C. difficile spores, mouse feces were collected and dispersed in an equal volume (w/v) of PBS containing protease cocktail, and the supernatants were collected by centrifugation and stored at −80° C. until use. To measure toxin-mediated cytotoxicity of fecal samples, the supernatants were diluted (final 100×) and filtered before adding to CT26 cell monolayers. Cell rounding was observed using a phase-contrast microscope. Goat anti-TcdA and -TcdB polysera (Techlab Inc. Blacksburg, Va.) were used to determine the specific activity of the C. difficile toxins.

### Example 3

### Cytokine Measurement

Cytokine concentration is determined in feces three times per week, and at necropsy from the large intestinal contents for IL-1β, IL-4, IL-6, IL-8, IL-10, IL-12, TNF-α, TGF-β, and IFN-γ using commercially available porcine cytokine ELISA kits (Invitrogen and R&D). Samples are stored at −20° C. until use. Fecal samples and large intestinal contents are diluted 1:2 to 1:10 with sterile PBS, depending on the consistency of the sample, thoroughly mixed using a vortex, then centrifuged, and the supernatant is added to reagent wells in the assay. The assay is performed following the manufacturer's instructions, and cytokine concentration is determined based on the standard curve (Steele, J. et al. 2010 J Infect Diseases 201:428).

### Example 4

### Antibody Titers and In Vitro Neutralizing Assay

The neutralizing titers against TcdA and TcdB for sera, intestinal lavage fluid, and fecal samples are determined. One week after the last immunization with the optimal dose of an atoxic protein (e.g., Tcd138), or toxoids, serum from each immunized mouse is collected. Sera from each group are pooled and neutralization of the cytotoxicity of either TcdA or TcdB is measured. Neutralizing titers and the optimal doses of the immunogens for parenteral immunization are determined. The calculated LD₅₀ᵢ toxin challenge doses are used to determine the level of protection induced by each immunogen. The protection level correlates with serum neutralizing titers, and, the atoxic protein described herein have significantly higher LD₅₀ᵢ and neutralizing titers than those of the toxoid. The LD₅₀ᵢ and neutralizing titers are used as references.

In mouse model, one day before an immunization and seven days after a previous immunization, serum samples from each immunized mouse were collected and IgG titers were measured using standard ELISA against purified each native or recombinant wild type holotoxins. In some treatments, the IgG titers were compared by using native toxins to coat ELISA plate with those using our recombinant toxins, and the results were essentially the same, showing that the antibody titers against His(6)-tag were negligible. In some treatments, serum antitoxin IgM and IgA, and fecal IgG and IgA were assessed by ELISA. To assess in vitro neutralizing activities of the serum samples, mouse intestinal epithelial cell line CT26 sensitive to both TcdA and TcdB was used. The neutralizing titer is defined as the reciprocal of the maximum dilution of serum that fails to block cell rounding induced by a standard concentration of a toxin. This concentration is four times the minimum dose of the toxin that causes essentially all CT26 cells to round after a 24-hour contact to the toxin. Wild type TcdA at 1.25 ng/ml or TcdB at 0.0625 ng/ml causes 100% of CT26 cells rounding after 24 hours of toxin treatment. Therefore, TcdA at 6 ng/ml or TcdB at 0.25 ng/ml was mixed with each of serially diluted serum samples which were then applied to CT26 cells. Cell rounding was observed using a phase-contrast microscope after 24 hours of incubation.

### Example 5

### Ultrasensitive Immunocytoxicity Assay

The current available assays to diagnose CDI, such as cytotoxin B assay, antibody-based immunoassays, GDH assay etc., have serious limitations. An ultrasensitive, tissue culture-based assay was developed based on recent findings and is here referred to as the immunocytotoxicity assay (He, X. et al. 2009a Infect Immun 77:2294-2303; He, X. et al. 2009b J Microbiol Methods 78:97-100; Herrmann et al., International patent application publication number WO 2010/006326 published Jan. 14, 2010 incorporated herein by reference hereby in its entirety). This assay detects the presence of less than 1 μg/ml of toxin in biological samples within four hours (He, X. et al. 2009 J Microbiol Methods 78:97-100, incorporated herein by reference). This assay was used to assess the systemic toxins in acute mouse and piglet models and the effect of antitoxins to reduce or eliminate the toxins.

The efficacy of parenteral immunization of the candidate vaccines (aTxAB and cTxAB) is evaluated and the optimal doses of immunization are determined to induce maximum antibodies to induce a protective response.

### Example 6

### Dose Optimization

The initial treatment used 5 μg total proteins per injection of the immunogens. Dose optimization of candidate atoxic proteins and toxoids was determined by determining the results of using doubled and halved optimized doses for parenteral immunization. If an adjuvant is used, e.g., mLT, the same amount of the adjuvant is mixed together with the immunogen before injection. For each dose and route of immunization, both systemic and mucosal IgG and IgA responses were monitored and neutralizing titers were measured. The lowest amount of antigen required to induce the highest level of serum and/or mucosal antibody response for each immunogen was established.

### Example 7

### Establishment of Challenge Dose of LD₅₀ᵢ

One week after the third immunization with the optimal dose of aTxAB, cTxAB, or toxoids, mice were challenged with doubling doses of LD₅₀ₙ, designated as doses causing death of 50% of naïve mice by wild type toxins. The dose that causes death of 50% of immunized mice were determined and designated as LD₅₀ᵢ. The LD₅₀ᵢ of each toxin for each immunogen was determined and the LD₅₀ᵢ of aTxAB were similar to that of cTxAB, both of which were significantly higher than those of toxoids.

### Example 8

### Mice, Cell Lines, and Toxins

Six- to 12-week-old BALB/c, CD1 and C57BL/6 mice were purchased from Jackson Laboratory (Bar Harbor, Me.) and housed in dedicated pathogen-free facilities. The mice were handled and cared for according to Institutional Animal Care and Use Committee (IACUC) guideline under protocols G950-07, G889-07, and G795-06. For evaluating systemic vaccination, ten mice per group (total four groups) were used and five mice were used for IP challenge by each toxin or toxin combination with two routes of immunization and three replicates of each treatment, with safety evaluation.

The murine colonic epithelial cell line CT26, the human colon epithelial cell lines HT-29 and HCT-8, and the monkey kidney cell line Vero were obtained from American Type Culture Collection (ATCC; Rockville, Mich.). Cells were maintained in Dulbecco's modified Eagle medium containing 10% fetal bovine serum, 100 U/ml penicillin, 100 μg/ml streptomycin, 2 mM L-glutamine and 1 mM sodium pyruvate. Native TcdA and TcdB toxins were purified from culture supernatants of toxigenic C. difficile strain VPI 10463 as previously described (Yang, G et al. 2008 BMC Microbiol 8: 192, incorporated by reference herein). Full-length wild type recombinant TcdA and TcdB proteins were purified from total crude extract of Bacillus megaterium as described in Yang, G et al. 2008 BMC Microbiol 8: 192. The biological activity of recombinant holotoxins was identical to their native forms (Yang, G et al. 2008 BMC Microbiol 8: 192). The highly purified recombinant toxins appeared as a single band on an SDS-PAGE gel and were devoid of detectable TLR2 and TLR4 ligand activity as determined by bioassays (He, X et al. 2009a. Infect Immun 77: 2294; Sun, X et al. 2009 Microb Pathog 46: 298, each of which is incorporated hereby in its entirety herein) and were used in Examples herein, unless otherwise specified.

Balb/C or C57BL/6 mice were immunized intraperitoneally (IP) with 5 μg of purified mutant toxins in PBS with alum as adjuvant for each injection. Control mice were injected PBS with alum. A total of 10 μg of protein per injection was administered, and mice were given three immunizations at 10 to 14 day intervals.

Systemic toxin challenge: Balb/c mice (four to six week old) were IP injected with wild type TcdA or TcdB (100 ng/mouse), or candidate atoxic protein (100 μg). Mice were observed closely for signs of disease and euthanized when they became moribund.

### Example 9

### Assessment of Binding of Immunogens to Mucosal Epithelium

A challenge facing mucosal antigen delivery is inefficient uptake of antigens by the mucosa. Although the receptor(s) are undefined, the receptor binding domain (RBD) of both C. difficile toxins contains multiple cell-wall binding repeats with high affinity to epithelial cells. Both aTxAB and cTxAB contain intact RBDs of C. difficile toxins, thus it is most likely that both immunogens can bind to epithelial cells with high affinity. To assess the binding of aTxAB and cTxAB to epithelial cells, the proteins are biotinylated before administration to mice using the method for biotinylation described in Keel, M. K. et al. 2007 Vet Pathol 44:814-822, in which neither the activities of the toxins, nor their binding to epithelium is affected. Six hours after mucosal (1N, SL, and oral) administration of the biotinylated aTxAB or cTxAB, mice are sacrificed and tissue sections are prepared for immunohistochemistry staining. To harvest sublingual mucosa, the floor of the mouth together with the tongue is excised en bloc from the mandible with thin curved scissors. The nasal mucosa is dissected following the method described in Eriksson, A. M. et al. 2004 J Immunol 173:3310-3319. To assess the binding of the immunogens to GI track after oral administration, the antral regions of the stomach and segments of the intestine from the immunized mice are collected. Specimens are fixed with paraformaldehyde and embedded in paraffin. For immunohistochemistry staining, the deparaffinized 6-μm-thick sections are pre-treated with biotin blocking kit before stained with HRP-conjugated avidin as described in Examples herein.

### Example 10

### Serum and Mucosal (Intestinal and Fecal) Antibody Response

The serum antibody response is analyzed as described in Examples herein. To examine the mucosal antibody response, intestinal lavage fluid (IL) is collected and fecal samples from mice and toxin-specific IgG and IgA are measured.

One day prior to each immunization and one week after the last immunization, fecal and IL samples are collected (Elson, C. O. et al. 1984 J Immunol Methods 67:101-108). Each mouse is kept on a 15 cm×15 cm wire mesh placed on top of a plastic petri dish containing 1 ml of a protease inhibitor cocktail. The mouse is restrained in a glass beaker on top of the wire mesh. To induce discharge of intestinal contents, four doses of 0.5 ml of lavage solution (25 mM NaCl, 40 mM Na₂SO₄, 10 mM KCl, 20 mM NaHCO₃, and 48.5 mM (162 g/l) polyethylene glycol (PEG) (average M_{w} 3350) are given at 15-minutes intervals using gavage. Thirty minutes after the last dose of lavage solution, the mice are given 0.1 mg of pilocarpine intraperitoneally. Intestinal contents (up to 0.5 ml) discharged over the next 20 minutes are collected in plastic tubes and kept frozen at −70° C. until use. Immediately before initiating the intestinal lavage procedure, two pieces of freshly voided feces are collected into 1.5-ml pre-weighed micro-centrifuge tubes. The feces are weighed before adding two volumes of PBS with protease inhibitor cocktail. Solid matter is suspended by extensive vortexing followed by centrifugation at 16,000×g for 10 minutes and the clear supernatants are stored at −70° C. until assayed.

Titers of antibodies specific for TcdA, TcdB IgG and IgA are determined by ELISA. Purified native TcdA or TcdB are used to coat the plates, which allows to minimize the cross reaction to His₆ Tag or possible contaminants in the immunogens. The detection limits of antitoxin IgA or IgG were set as two times of OD405 over background in the well with the lowest dilution.

### Example 11

### Histopathological Analysis

Histopathological analysis was performed to evaluate mucosal damage and inflammation induced by the toxins. Resected colon or cecum tissues were fixed in 4% formaldehyde buffered with PBS and then embedded with paraffin. De-paraffinized 6-μm-thick sections were stained with hematoxylin and eosin (H&E) for histological analysis.

### Example 12

### Protection Against Mucosal Challenge with the Toxins

Rabbit antisera specific for TcdA were observed to block TcdA-induced intestinal inflammation and tissue damage as was shown in mouse ileal loop model. Mucosal IgA and IgG antibodies against toxins are generated and ability to protect mice against toxin-mediated destruction of the mucosa is examined. Because pure TcdB has no enterotoxicity and does not induce mucosal inflammation and tissue destruction in mice, only mucosal protection against TcdA is examined using ileal loop model. The ability of mucosal immunization of atoxic proteins described herein to induce mucosal protection against TcdB and against TcdA in orally challenged C. difficile mouse and piglet infection models is also examined.

The ileal loop models are used one week after the third immunization. In normal Balb/c mice, a high dose of 50 μg of wild type TcdA was observed to cause substantial fluid accumulation and mucosal damage, whereas a lower dose of 10 μg of TcdA caused only mild mucosal destruction within four hours of toxin treatment. Therefore, these two doses are used. Three 3-cm loops are ligated in each mouse and injected with 10 or 50 μg of wild TcdA, or an equal volume of PBS (100 μl). The same treatments are performed in control placebo treated mice. The toxin-induced fluid accumulation is quantitated, and data are analyzed using one-way ANOVA. P values between groups are determined using Bonferoni's multiple comparison test.

In addition to assessing the fluid accumulation, the pathological signs, such as neutrophil infiltration and villus damage, are evaluated histologically and compared between the groups. Histopathological and neutrophil myeloperoxidase (MPO) activity assays are performed to evaluate mucosal damage and neutrophil infiltration. The loops are collected, and the resected intestines are fixed in 4% formaldehyde buffered with PBS and then embedded with paraffin. Deparaffinized 6-μm-thick sections are stained with haematoxylin and eosin (H&E) for histological analysis, and the tissue injuries are blindly scored by a histologist. Histological grading criteria used are as follows: 0, minimal infiltration of lymphocytes, plasma cells, and eosinophils; 1+, mild infiltration of lymphocytes, plasma cells, neutrophils, and eosinophils plus mild congestion of the mucosa with or without hyperplasia of gut-associated lymphoid tissue; 2+, moderate infiltrations of mixed inflammatory cells, moderate congestion and edema of the lamina propria, with or without goblet cell hyperplasia, individual surface cell necrosis or vacuolization, and crypt dilatation; 3+, severe inflammation, congestion, edema, and hemorrhage in the mucosa, surface cell necrosis, or degeneration with erosions or ulcers (Torres, J. F. et al. 1995 Infect Immun 63:4619-4627). To measure MPO activity in the samples, a portion of the resected ileum is freeze-dried and homogenized in 1 ml of 50 mM potassium phosphate buffer with 0.5% hexadecyl trimethyl ammonium bromide and 5 mM EDTA. The tissues are disrupted with sonication and freeze-thaw cycles, and centrifuged. MPO activity in the supernatant is determined using substrate o-phenylenediamine in 0.05% of H₂O₂, and absorbance is measured at 490 nm using a plate reader.

Mucosal vaccination is expected to protect against TcdA challenge in the intestine. TNF-α was observed to play a crucial role in C. difficile toxin-induced intestinal inflammation. TcdA induced a complete destruction of villi and massive infiltration of immune cells in wild type mice, and TNFR KO mice showed mild damage of intestinal villi and moderate infiltration of immune cells in response to TcdA.

### Example 13

### Statistical Analysis of Piglet Model

In piglet model, the data obtained from treatments are analyzed using a non-parametric test (Wilcoxon analysis) following ANOVA using SigmaStat v. 3.1 (Systat Software, Inc.). For four groups, including a control group, for a power of 0.8 and alpha=0.05, a sample size of 5-118 is required depending on level of T² desired. Seven animals/group (n=7) were used for challenge studies involving evaluation of vaccine candidates. Survival curves are compared and analyzed by Log-rank (Mantel-Cox) Test or Gehan-Breslow-Wilcoxon Test using GraphPad Prism software.

These data are complemented with Group Pair-Wise Comparisons (Levene's/ANOVA-Dunnett's/Welch's). The Levene's test is used to assess homogeneity of group variances for each specified endpoint and for all collection intervals. If Levene's test is not significant (p>0.01), a pooled estimate of the variance (Mean Square Error or MSE) is computed from a one-way analysis of variance (ANOVA) and utilized by a Dunnett's comparison of each treatment group with the two control groups. If Levene's test is significant (p<0.01), comparisons with the control group are made using Welch's t-test with a Bonferroni correction. Results of pair-wise comparisons are reported at the 0.05 and 0.01 significance levels. Endpoints are analyzed using two-tailed tests unless indicated otherwise.

### Example 14

### Technological Advantages

Both systemic and mucosal immunity provide protection against enteric pathogens and pathogenic products such as toxins (Byrd, W. et al 2006 FEMS Immunol Med Microbiol 46:262-268; Huang, C. F. et al. 2008 J Pediatr Gastroenterol Nutr 46:262-271; Lucas, M. E. et al. 2005 N Engl J Med 352:757-767; Perez, J. L. et al. 2009 Vaccine 27:205-212). Because TcdA and TcdB are virulent factors for C. difficile, an antitoxin antibody preparation can convey full protection from oral C. difficile challenge in animals (Kink, J. A. et al. 1998 Infect Immun 66:2018-2025; Lyerly, D. M. et al. 1991 Infect Immun 59:2215-2218). Antibodies against both toxins, but not against TcdA or TcdB alone, protect toxigenic C. difficile infection in hamster model (Fernie, D. S. et al. 1983 Dev Biol Stand 53:325-332; Kim, P. H. et al. 1987 Infect Immun 55:2984-2992; Libby, J. M. et al. 1982 Infect Immun 36:822-829). An evaluation of the routes of delivery of toxoid vaccine in hamsters assessing protection from both lethal disease and diarrhea have found that a combination of mucosal and parental immunization provided complete protection from death and diarrhea, suggesting that induction of both systemic and mucosal immunity was necessary for optimal protection (Torres, J. F. et al. 1995 Infect Immun 63:4619-4627).

In humans, a higher level of antitoxins in serum is associated with less severe disease and less frequent relapse (Kyne, L. et al. 2000 N Engl J Med 342:390-397). Following symptomatic infection, most individuals develop anti-TcdA and anti-TcdB antibodies in serum (Aronsson, B. et al. 1985 Infection 13:97-101; Viscidi, R. et al. 1983 J Infect Dis 148:93-100), including toxin-neutralizing IgA in serum as well as in stool (Johnson, S. et al. 1995 Infect Immun 63:3166-3173), and this systemic and mucosal antibody response appears to be associated with protection from subsequent infection.

Clostridium difficile-associated diarrhea and enteric inflammatory diseases are caused primarily by two secretory toxins. A vaccine (mucosal and/or parenteral delivery) is proposed herein to reduce the incidence and severity of Clostridium difficile infection (CDI), using recently expressed atoxic C. difficile toxin proteins in an endotoxin-free Bacillus megaterium system (Yang, G. et al. 2008 BMC Microbiol 8:192, incorporated herein by reference). Candidate vaccines are here evaluated, and results show effective immunization specific for C. difficile. Protection against CDI has been shown to be mediated through systemic and mucosal antibodies against the two key toxins, although other virulence attributes are known to exist which may also contribute to the manifestation of CDI.

The focus was on designing a vaccine that targets both TcdA and TcdB, in order to elicit strong systemic and mucosal immunity. Atoxic proteins (e.g., Tcd138, derivates, and homologs thereof) were found to be superior to toxoid or fragments thereof. Without being limited to any theory or mode of action the Examples herein showed that the atoxic proteins are useful for generating a full spectrum of neutralizing antibodies. Unlike chemical-detoxified toxoid, or fragments that contain a small portion of TcdA, these atoxic holotoxins generated by point mutations maintain the same adjuvant activity, antigenicity, and affinity to mucosal epithelium as do native toxins, thus induce superior protective immunity than toxoid and wider spectrum of antibodies than fragments.

Atoxic protein vaccination was shown to induce antibody responses against a wide-spectrum of epitopes and potent protective immunity superior to toxoid, and Tcd138 protein immunization induced antibody and protective responses against the toxins. People at high risk of C. difficile infection, such as under antibiotic treatment and/or hospitalization, are logical targets for prophylactic vaccination. The chimeric Tcd138 vaccination for example induced potent protection in mice against lethal challenge with both TcdA and TcdB.

The ability of these atoxic recombinant proteins was evaluated to induce protective antibody responses following parenteral immunization followed by challenge with wild type toxins, followed by the evaluation of several regimens of mucosal immunizations (oral, intranasal and sublingual) designed to induce protection against systemic and mucosal challenges with wild type toxins. The protective efficacy of the various immunization regimens developed was tested in the mouse acute infection model, and the most efficient immunization method resulting from the mouse infection studies undergo preclinical evaluation in the chronic piglet model of CDI.

These Examples show that a novel C. difficile candidate vaccine was developed that is productively expressed in a safe, environmental, and endotoxin-free bacterial host, B. megaterium (Vary P S et al. 2007 Applied microbiology and biotechnology 76: 957; Yang, G et al. 2008 BMC Microbiol 8: 192). Compared to native toxins purified from C. difficile culture, the recombinant Tcd138 protein is significantly easier and cheaper to purify in a large quantity. It is a single antigen maintaining a toxin-like conformation and capable of inducing potent neutralizing antibodies against the both toxins. This candidate vaccine not only induces full and long-lasting protection against C. difficile-associated morbidity and mortality, but also rapid protection against primary and recurrent CDI. Examples herein show that both primary and recurrent CDI can be prevented by systemic antibodies through parenteral vaccination.

The major virulence factors of toxigenic C. difficile are the two large secreted glucosyltransferase protein toxins A (TcdA) and B (TcdB). Examples herein engineered and constructed a designated “mTcd138” or “Tcd138” which is a fusion protein comprising the N-terminus of TcdB and the receptor binding domain (RBD) of TcdA. Two point mutations were engineered in Tcd138 in the N-terminus of TcdB in Examples herein that essentially eliminates the glucosyltransferase activity. The point mutations ensure that Tcd138 is atoxic. Tcd138 was expressed in a Bacillus megaterium system with a yield of four milligrams per liter (mg/L).

Without being limited by any particular theory or mechanism of action, it is here envisioned that greater yields could be obtained in other expression systems such as E. coli systems or mammalian systems.

Mice subjects were immunized three times at a 14-day interval with ten micrograms (μg) of Tcd138 protein. Immunization with Tcd138 induced a rapid and potent serum IgG response against both TcdA toxin and TcdB toxin, and protected subjects from systemic challenge with at least a two-fold lethal dose of TcdA or TcdB and from infection of an epidemic strain of C. difficile. In various embodiments, subjects administered the Tcd138 protein were more greatly protected from TcdA and TcdB than subjects administered cTxAB protein. The chimeric protein cTxAB is described in Feng et al., international application number PCT/US2010/058701 filed Dec. 2, 2010, which is incorporated by reference herein in its entirety. The protection induced by Tcd138 vaccination is long-lasting, and the immunized subjects are fully protected against a C. difficile infection and challenge.

1. A composition for eliciting an immune response specific for a Clostridium difficile toxin, the composition comprising an atoxic protein or a source of expression of the protein, wherein the protein contains a glucosyltransferase domain (GT), a cysteine proteinase domain (CPD), a receptor binding domain (RBD), and a first amino acid sequence of the RBD derived from a TcdA protein and a second amino acid sequence of the GT and CPD from a TcdB protein, and a mutation, wherein the protein is atoxic.

2. The composition according to claim 1, wherein the atoxic recombinant protein comprises a mutation in at least one C. difficile protein selected from the group of a TcdA protein and a TcdB protein, and retains native protein conformation, wherein toxicity of the protein is reduced at least: about 10-fold to about 1,000-fold, or about 1,000-fold to about 10,000-fold, or about 10,000-fold to about 10 million-fold compared to toxicity of wild-type Clostridium toxin.

3. The composition according to claim 2, wherein the protein comprises Tcd138.

4. The composition according to claim 3, wherein the mutation is located in the GT domain of the TcdB protein.

5. The composition according to claim 1, wherein the atoxic recombinant protein further comprises a purification tag.

6. The composition according to claim 1, wherein the source of the protein is selected from an expression system of at least one of: a Gram-positive bacterial cell; a yeast cell; a bird cell; and a mammalian cell.

7. The composition according to claim 6, wherein the Gram positive bacterial cell comprises a Bacillus.

8. (canceled)

9. The composition according to claim 1 further comprising at least one of an adjuvant and a pharmaceutically acceptable carrier.

10. The composition according to claim 1, wherein the source of the protein comprises a vector carrying a nucleotide sequence encoding the protein.

11. The composition according to claim 1, wherein the vector comprises a viral vector or a plasmid.

12. The composition according to claim 11, wherein the viral vector is derived from a genetically engineered genome of at least one virus selected from the group consisting of adenovirus, adeno-associated virus, a herpesvirus, and a lentivirus.

13. (canceled)

14. A method of eliciting an immune response specific for a Clostridium difficile toxin in a subject, the method comprising:
contacting the subject with a composition comprising an atoxic protein or a source of expression of the protein, wherein the protein contains a glucosyltransferase domain (GT), a cysteine proteinase domain (CPD), a receptor binding domain (RBD), and a first amino acid sequence of the RBD derived from a TcdA protein and a second amino acid sequence derived of the GT and CPD from a TcdB protein, and a mutation, wherein the protein is atoxic and elicits the immune response specific for the Clostridium difficile toxin in the subject.

15. The method according to claim 14 further comprising prior to contacting, engineering a vector carrying the nucleotide sequence encoding the protein.

16. The method according to claim 15, wherein engineering further comprises expressing the protein in a cell.

17. The method according to claim 15, wherein engineering comprises obtaining the mutation in at least one of: a TcdA nucleic acid sequence encoding the TcdA amino acid sequence, and a TcdB nucleic acid sequence encoding the TcdB amino acid sequence.

18. The method according to claim 14, wherein the mutation comprises at least one selected from the group consisting of: a substitution, a deletion, and an addition.

19. The method according to claim 14, wherein the source of expression of protein is at least one selected from the group consisting of: a nucleic acid vector with a gene encoding the protein; a viral vector with a gene encoding the protein; and a cell that expresses the protein.

20. The method according to claim 14, wherein contacting the subject further comprises administering the protein by a route selected from at least one of the group consisting of intravenous, intramuscular, intraperitoneal, intradermal, mucosal, subcutaneous, sublingual, intranasal and oral.

21. (canceled)

22. A method of producing a recombinant atoxic Clostridium difficile toxin protein, the method comprising:
constructing a vector carrying a nucleotide sequence encoding the protein, wherein the protein comprises a glucosyltransferase domain (GT), a cysteine proteinase domain (CPD), a receptor binding domain (RBD), a first amino acid sequence of the RBD from a TcdA protein and a second amino acid sequence of the GT and CPD from a TcdB protein, and a mutation, wherein the protein is atoxic;
contacting a cell with the vector under conditions suitable to transformation or transduction of the cell; and,
selecting a transformant carrying the selectable marker and expressing the recombinant atoxic Clostridium toxin protein.

23. The method according to claim 22, wherein the cell is a protoplast selected from the group of: B. megaterium, B. subtilis, B. thuringiensis, B. cereus, and B. licheniformis.

24. The method according to claim 22, wherein constructing the vector comprises combining a first nucleic acid sequence encoding the TcdA first amino acid sequence and a second nucleic acid sequence encoding the TcdB second amino acid sequence, and wherein sequence is operably linked to a regulatory region comprising a promoter.

25. The method according to claim 24, wherein the protein comprises a plurality of mutations selected from at least one of: a substitution, a deletion, and an addition.

26. The method according to claim 25, wherein the mutation is located in the GT domain of the TcdB protein.

27. The method according to claim 22, wherein the mutation of the protein comprises a deletion of a transmembrane domain.

28-31. (canceled)

32. The composition according to claim 1, which elicits an immune response specific for Clostridium difficile toxins TcdA and TcdB, the composition comprising a protein or a source of expression of the protein, the protein comprising a glucosyltransferase domain (GT) and a cysteine proteinase domain (CPD) of the TcdB, a receptor binding domain (RBD) of the TcdA, and at least two point mutations in the GT, wherein the protein is atoxic.

33-60. (canceled)
